(19) United States
(12) Patent Application Publication
(10) Pub. No.: US 2026/0258253 A1
(43) Pub. Date: Sep. 3, 2026

(54) PHOTOINITIATORS FOR PHOTOCURABLE COMPOSITIONS

(71) Applicant: Covestro (Netherlands) B.V., Geleen (NL)

(72) Inventors: Johan Franz Gradus Antonius Jansen, Geleen (NL); Guido Hensen, Oirsbeek (NL)

(21) Appl. No.: 18/863,380

(22) PCT Filed: May 22, 2023
(86) PCT No.: PCT/EP2023/063641

§ 371 Date: Nov. 6, 2024

(30) Foreign Application Priority Data
May 24, 2022 (EP) 22175249.6

(51) Int. Cl. C09D 4/00; C07C 69/33; C09D 11/101; C09D 11/102; C09D 11/107; C09D 135/02; C09D 175/14

(52) U.S. Cl. CPC C09D 4/00; C07C 69/33; C09D 11/101; C09D 11/102; C09D 11/107; C09D 135/02; C09D 175/14

(57) ABSTRACT

The present invention relates to an oligomeric photoinitiator according to formula (I)

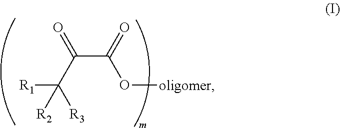

wherein
m is equal to or greater than 3,
R₁, R₂ and R₃ are independently selected from H or from linear or branched C₁-C₆ alkyl, and wherein the oligomeric photoinitiator has a weight-average molecular weight M_{w} as determined by GPC (THF) higher than 1000 g/mol.

The present invention relates to oligomeric photoinitiators that are useful as photoinitiators in free radical curable compositions, such as free radical curable coating and ink compositions. The invention further relates to free radical curable compositions comprising such an oligomeric photoinitiator. Further, the invention relates to objects coated with such a free radical curable compositions.

Nowadays coatings are used every day. Examples are items like coatings for transparent food packages, thin foils, paints, and car part finishes. UV curing or free radical photopolymerization is the fastest growing curing technique, with a continuously increasing number of applications. UV curing saves energy and reduces or eliminates solvent emission in comparison with solvent-based systems because most radiation-curable formulations are 100% solid formulations containing reactive oligomers and diluents. The mechanical properties of the cured formulations are generally determined by the oligomers and diluents. Photoinitiators are one of the key components for every photopolymerizable formulation as they generate upon light exposure radicals which trigger the polymerization. A major drawback for most photopolymerizable formulations is the amount of unreacted photoinitiator molecules remaining in the cured polymer material. The high conversion rates in a very short time imply that only a few initiator molecules are able to covalently bind to the polymer network. Ways to solve this are for example irradiating for a very long period of time or using small amounts of initiator. Both ways are not suitable for industrial scale applications as short exposure times ensure the required high throughput. A reasonable conversion is normally provided by using an increased percentage of photoinitiator leading to aforementioned increased amounts of unreacted photoinitiator in the cured formulations. Next to migrating out of the cured formulation, the remaining photoinitiators can cause yellowing, generate a variety of byproducts like for example benzaldehyde which can migrate out of the cured formulations as well. Migration is especially problematic when protective and decorative coatings are applied in the field of food packaging, health care, and other all day use items, which can come in contact with humans.

Benzophenone and its derivatives are more commonly used as photoinitiators in the packaging area. However, for benzophenone there is sufficient evidence in experimental animals for its carcinogenity and it is possible carcinogenic to humans (International Agency for Research on Cancer, “some chemicals present in industrial and consumer products, food and drinking water”, vol 101 p 285-301).

In fact one study (R. Z. Liu, S. A. Mabury,” First Detection of Photoinitiators and Metabolites in Human Sera from United States Donors” Environmental Science and Technology, vol 52(17), p 10089-10096 2018.) revealed that photoinitiators and coinitiators were also found in every single blood serum illustrating that photoinitiators and their photoproducts are omnipresent contaminants.

This illustrates the need for free radical curable formulations in which alternative especially oligomeric photo initiators are used. By using oligomeric photo initiators, the migratability is dramatically reduced. An example of such an oligomeric photo initiator is given in WO2021/259924. However, in this application the basis for the oligomeric photo initiator are still benzophenone derivatives.

A very elegant alternative to benzophenone derivatives has been described in US2020/0231531, i.e. a linear polymer based on α-ketoglutaric acid and hexane diol. However, as will be demonstrated, the reactivity of such a system is not sufficient. Consequently there is a need for oligomeric photoinitiators with increased reactivity.

The object of the present invention is to provide oligomeric photoinitiators with increased reactivity in free radical curing processes.

According to the invention, there is provided an oligomeric photoinitiator according to formula (I)

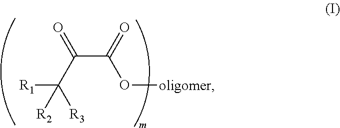

wherein
m is equal to or greater than 3,
R₁, R₂ and R₃ are independently selected from H or from a linear C₁-C₆ alkyl or from a branched C₁-C₆ alkyl, and
wherein the oligomeric photoinitiator has a weight-average molecular weight M_{w} as determined by GPC (THF) higher than 1000 g/mol, whereby the weight-average molecular weight M_{w} is determined as described in the description.

It has surprisingly been found that the photoinitiators according to the invention have increased reactivity in free radical curable compositions. The photoinitiators according to the invention can in particular advantageously be applied in free radical curable coating and ink compositions wherein a low migration of chemical species is required.

For all upper and/or lower boundaries of any range given herein, the boundary value is included in the range given, unless specifically indicated otherwise. Thus, when saying from x to y, means including x and y and also all intermediate values.

The oligomeric photoinitiator according to formula (I) comprises m (m is equal to or greater than 3) terminal functional groups according to formula (Ia)

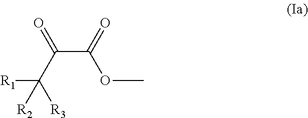

Preferably m is equal to or greater than 4, more preferably m is equal to or greater than 5, even more preferably m is equal to or greater than 6, even more preferably m is equal to or greater than 7, even more preferably m is equal to or greater than 8.

Preferably, R₁, R₂ and R₃ are independently selected from H or CH₃. More preferably R₁, R₂ and R₃ are H. In case R₁, R₂ and R₃ are H, the terminal functional groups according to formula (Ia) are derived from pyruvic acid with formula

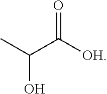

The oligomeric photoinitiator can be obtained in a process that comprises at least the following steps:
(1) providing a core oligomer having at least m (m≥3) terminal OH functional groups,
(2) (trans)esterification of m terminal OH functional groups of the core oligomer with a compound according to formula (Ib):

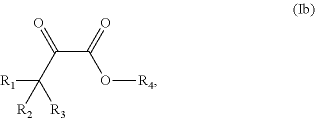

wherein R₄ is H or a C₁-C₄ alkyl, preferably a C₁-C₃ alkyl; and R₁, R₂ and R₃ are as described above.

The (trans)esterification is usually effected with a catalyst known to the skilled in the art, for example an acid catalyst such as for example methane sulfonic acid or sulfuric acid. The formed water (for R₄=H) or the formed small alcohol (for R₄=C₁-C₄ alkyl) (methanol, ethanol, . . . ) can be removed physically by the help of an entrainer such as toluene, a nitrogen gas stream, or vacuum. Preferably, R₄=H.

An epoxy resin, such as for example Epikote™ 828 obtainable from Hexion, can be used to scavenge the acid catalyst.

For incorporation of a multitude of functional groups (including at least the m terminal functional groups with formula (Ia)), the core oligomer is preferably a branched oligomer, more preferably a highly branched oligomer, even more preferably a hyperbranched oligomer. Using branched or highly branched or hyper branched oligomers as core oligomer has the advantage that they can have a multitude of terminal groups, preferably hydroxy terminal groups which can be readily modified resulting in a structure according to Formula (I). Under highly branched oligomers are understood in the context of the present invention oligomers having a branched structure and a high density of functional groups with formula (Ia). Under hyperbranched oligomers are understood in the context of the present invention oligomers having a branched structure and an even higher density of functional groups with formula (Ia).

The inventors have further discovered that it might be beneficial that the oligomeric photoinitiator comprises from 1 to 10 groups according to Formula (II):

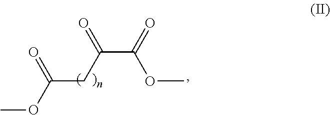

wherein n is from 0 to 6, preferably n is 2 or 3, more preferably n is 2.

In case n is 3, the groups according to formula (II) are derived from a compound with formula:

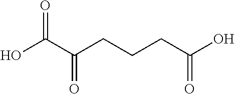 [SMILES: O=C(O)CCCC(=O)C(=O)O]

In case n is 2, the groups according to formula (II) are derived from 2-ketoglutaric acid with formula:

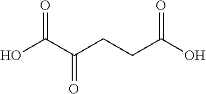

### Core Oligomers

The core oligomer can be a polyether, a polyester, a polycarbonate, a polyamide, or a polyacrylic oligomer, and may optionally further comprises urethane linkages. Preferred core oligomers are polyether oligomers optionally further comprising urethane linkages or polyester oligomers optionally further comprising urethane linkages.

Polyether core oligomers are for example be derived from ethoxylated or propoxylated glycerol, ethoxylated or propoxylated trimethylopropane, ethoxylated or propoxylated pentaerythritol, ethoxylated or propoxylated ditrimethylol propane and/or ethoxylated or propoxylated dipentaerythritol.

Polyester core oligomers can for example be derived from di- and/or tri acids or their esters, together with polyvalent alcohols such as di, tri, tetrols, hexols and/or octols. Examples of di-acids are adipic acid, succinic acid, ketoglutaric acid and malonic acid. By employing for example ketoglutaric acid or malonic acid, structures according to formula (II) are incorporated in the core oligomer. An example of a triacid is trimellitic acid (or anhydride). Suitable alcohols can be diols like butanediol, hexanediol, ethylene glycol, diethylene glycol, propylene glycol, dipropylene glycol; triols like glycerol, like trimethylol propane and their alkoxylated versions; tetrols like pentaerythritol and di(trimethylol propane) and their alkoxylated versions; hexols like dipentaerythritol and sorbitol and their alkoxylated versions; octols like sucrose and its alkoxylated version.

Obviously hydroxyacids and/or esters can also be employed for obtaining a polyester core oligomer, examples are linear hydroxy acids like lactic acid, gamma-hydroxy butyric acid, caprolactone, branched hydroxy acids like citric acid or 2,2-dimethylol propionic acid. The latter might be used to prepare branched core oligomers of which Boltorn™ H2004 and H311 are examples. Branched amide containing cores are for example Hybrane™ cores Polyester core oligomers can also be obtained via the so called anhydride oxetane reaction. An example of such a core oligomer can be obtained by using trimellitic anhydride in combination with trimethyloloxetane.

As mentioned before, the core oligomer can also be an acrylic oligomer having multiple hydroxyl groups. These hydroxy functional oligomers can for example be prepared via radical polymerization from for example hydroxy ethyl (meth)acrylate. The skilled artisan will recognize that other hydroxy functional unsaturated compounds can be used as well. Examples are hydroxybutyl monovinyl ether, hydroxyethyl maleimide, caprolactone acrylate, ethoxylated or propoxylated acrylic acid. The hydroxy functional unsaturated compounds can be oligomerized/polymerized on themselves or copolymerized. For that copolymerization a wide plethora of other unsaturated compounds are available such as for example various (meth) acrylates, styrene, acrylonitrile, and vinyl ethers.

In view of reduced migrateability, the molecular weight of the oligomeric photoinitiator according to the invention is preferably higher than 1250 g/mol, more preferably higher than 1500 g/mol.

The oligomeric photoinitiator according to the invention preferably comprises free radical polymerizable groups. These free radical polymerizable groups are preferably selected from the group consisting of methacrylates, acrylates, vinyl amides, vinyl ethers, vinyl esters, fumarates, itaconates, maleates and any combination thereof. Most preferably the free radical polymerizable groups are methacrylate and/or acrylate end groups.

The present invention further relates to a free radical curable composition comprising the oligomeric photoinitiator as described herein above.

The amount of oligomeric photoinitiator according to the invention in the composition can vary within wide ranges like from 0.001 wt. % up to 99 wt. %. Preferably the amount of oligomeric photoinitiator according to the invention is between 0.05 wt. % and 30 wt. %, relative to the entire weight of the free radical curable composition. The amount of oligomeric photoinitiator according to the invention is preferably higher than 0.1 wt. %, more preferably higher that 0.5 wt. % or higher than 1 wt. % or higher than 2 wt. % and preferably lower than 20 wt. % or lower than 15 wt. % or lower than 10 wt. %, relative to the entire weight of the free radical curable composition. Depending on the amount of free radical polymerizable groups in the oligomer photoinitiator, higher amounts of oligomeric photoinitiator can be suitably employed as well. For example when the photoinitiator according to the invention comprises 2 or more acrylate end groups, the photoinitator can be considered a free radical curable oligomer by itself and can be used in amounts ranging from 20 to 70 wt. %.

Although in case the oligomeric photoinitiator comprises free radical polymerizable groups, other free radical polymerizable compounds are not required, the free radical curable composition preferably further comprises one or more free radical curable oligomers having one or more free radical curable ethylenically unsaturated groups, preferably having one or more (meth)acroyl groups or vinyl groups. The one or more oligomers having one or more free radical curable ethylenically unsaturated groups preferably have a number average molecular weight (Mₙ) equal to or higher than 600 g/mol, in particular from 800 to 15,000 g/mol, more particularly from 1,000 to 5,000 g/mol, whereby the number-average molecular weight Mₙ is determined as described herein below.

The one or more oligomers having one or more free radical curable ethylenically unsaturated groups are present in the free radical curable composition in an amount of preferably at least 10 wt. %, or at least 15 wt. %, or at least 20 wt. %, and at most 80 wt. %, or at most 75 wt. %, or at most 70 wt. %, relative to the entire weight of the free radical curable composition.

Preferably the free radical curable oligomers are selected from the group consisting of urethane (meth)acrylates, epoxy (meth)acrylates, polyester (meth)acrylates, polyether (meth)acrylates and any mixture thereof.

The (meth)acrylate-functionalized oligomer may be selected in order to enhance the flexibility, strength and/or modulus, among other attributes, of a cured polymer prepared using the free radical curable composition of the present invention. The (meth)acrylate functionalized oligomer may have 1 to 18 (meth)acrylate groups, in particular 2 to 6 (meth)acrylate groups, more particularly 2 to 6 acrylate groups. The (meth)acrylate functionalized oligomer may have a number average molecular weight equal of more than 600 g/mol, in particular from 800 to 15,000 g/mol, more particularly from 1,000 to 5,000 g/mol. In particular, the (meth)acrylate-functionalized oligomers may be selected from the group consisting of (meth)acrylate-functionalized urethane oligomers (sometimes also referred to as “urethane (meth)acrylate oligomers,” “polyurethane (meth)acrylate oligomers” or “carbamate (meth)acrylate oligomers”), (meth)acrylate-functionalized epoxy oligomers (sometimes also referred to as “epoxy (meth)acrylate oligomers”), (meth)acrylate-functionalized polyether oligomers (sometimes also referred to as “polyether (meth)acrylate oligomers”), (meth)acrylate-functionalized polydiene oligomers (sometimes also referred to as “polydiene (meth)acrylate oligomers”), (meth)acrylate-functionalized polycarbonate oligomers (sometimes also referred to as “polycarbonate (meth)acrylate oligomers”), and (meth)acrylate-functionalized polyester oligomers (sometimes also referred to as “polyester (meth)acrylate oligomers”), acrylic (meth)acrylate oligomers and mixtures thereof. Preferably, the (meth)acrylate-functionalized oligomer comprises a (meth)acrylate-functionalized urethane oligomer, more preferably an acrylate-functionalized urethane oligomer. Advantageously, the (meth)acrylate-functionalized oligomer comprises a (meth)acrylate-functionalized urethane oligomer having two (meth)acrylate groups, more preferably an acrylate-functionalized urethane oligomer having two acrylate groups. Exemplary polyester (meth)acrylate oligomers include the reaction products of acrylic or methacrylic acid or mixtures or synthetic equivalents thereof with hydroxyl group-terminated polyester polyols. The reaction process may be conducted such that all or essentially all of the hydroxyl groups of the polyester polyol have been (meth)acrylated, particularly in cases where the polyester polyol is difunctional. The polyester polyols can be made by polycondensation reactions of polyhydroxyl functional components (in particular, diols) and polycarboxylic acid functional compounds (in particular, dicarboxylic acids and anhydrides). The polyhydroxyl functional and polycarboxylic acid functional components can each have linear, branched, cycloaliphatic or aromatic structures and can be used individually or as mixtures. 35

Examples of suitable epoxy (meth)acrylate oligomers include the reaction products of acrylic or methacrylic acid or mixtures thereof with an epoxy resin (polyglycidyl ether or ester). The epoxy resin may, in particular, by selected from bisphenol A diglycidyl ether, bisphenol F diglycidyl ether, bisphenol 6 diglycidyl ether, brominated bisphenol A diglycidyl ether, brominated bisphenol F diglycidyl ether, epoxy novolak resin, hydrogenated bisphenol A diglycidyl ether, hydrogenated bisphenol F diglycidyl ether, hydrogenated bisphenol S diglycidyl ether, 3,4-epoxycyclohexylmethyl 3′,4′epoxycyclohexanecarboxylate, 2-(3,4-epoxycyclohexyl-5,5-spire-3,4-epoxy)cyclohexane-1,4-dioxane, bis(3,4-epoxycyclohexylmethyl)adipate, vinylcyclohexene oxide, 4-vinylepoxycyclohexane, bis(3,4-epoxy-6-methylcyclohexylmethyl)adipate,3,4-epoxy-6-methylcyclohexy 1-3′,4uepoxy-6u methylcyclohexanecarboxylate, methylenebis(3,4-epoxycyclohexane), dicyclopentadiene diepoxide, di(3,4-epoxycyclohexylmethyl) ether of ethylene glycol, ethylenebis(3, 4-epoxycyclohexanecarboxylate), 1,4-butanediol diglycidyl ether, 1,6-hexanediol diglycidyl ether, glycerol triglycidyl ether, trimethylolpropane triglycidyl ether, polyethylene glycol diglycidyl ether, polypropylene glycol diglycidyl ether, polyglycidyl ethers of a polyether polyol obtained by the addition of one or more alkylene oxides to an aliphatic polyhydric alcohol such as ethylene glycol, propylene glycol, and glycerol, diglycidyl esters of aliphatic long-chain dibasic acids, monoglycidyl ethers of aliphatic higher alcohols, monoglycidyl ethers of phenol, cresol, butyl phenol, or polyether alcohols obtained by the addition of alkylene oxide to these compounds, glycidyl esters of higher fatty acids, epoxidized soybean oil, epoxybutylstearic acid, epoxyoctylstearic acid, epoxidized linseed oil, epoxidized polybutadiene, and the like.

Suitable polyether (meth)acrylate oligomers include, but are not limited to, the condensation reaction products of acrylic or methacrylic acid or synthetic equivalents or mixtures thereof with polyetherols which are polyether polyols (such as polyethylene glycol, polypropylene glycol or polytetramethylene glycol). Suitable polyetherols can be linear or branched substances containing ether bonds and terminal hydroxyl groups. Polyetherols can be prepared by ring opening polymerization of cyclic ethers such as tetrahydrofuran or alkylene oxides (e.g., ethylene oxide and/or propylene oxide) with a starter molecule. Suitable starter molecules include water, polyhydroxyl functional materials, polyester polyols and amines.

Polyurethane (meth)acrylate oligomers (sometimes also referred to as “urethane (meth)acrylate oligomers”) suitable for use in the curable compositions of the present invention include urethanes based on aliphatic, cycloaliphatic and/or aromatic polyester polyols and polyether polyols and aliphatic, cycloaliphatic and/or aromatic diisocyanates and capped with (meth)acrylate end-groups. Suitable polyurethane (meth)acrylate oligomers include, for example, aliphatic polyester-based urethane di- and tetra-acrylate oligomers, aliphatic polyether-based urethane di- and tetra-acrylate oligomers, as well as aliphatic polyester/polyether-based urethane di- and tetraacrylate oligomers. The polyurethane (meth)acrylate oligomers may be prepared by reacting aliphatic, cycloaliphatic and/or aromatic polyisocyanates (e.g., diisocyanate, triisocyanate) with OH group terminated polyester polyols, polyether polyols, polycarbonate polyols, polycaprolactone polyols, polyorganosiloxane polyols (e.g., polydimethylsiloxane polyols), or polydiene polyols (e.g., polybutadiene polyols), or combinations thereof to form isocyanate-functionalized oligomers which are then reacted with hydroxyl-functionalized (meth)acrylates such as hydroxyethyl acrylate or hydroxyethyl methacrylate to provide terminal (meth)acrylate groups. For example, the polyurethane (meth)acrylate oligomers may contain two, three, four or more (meth)acrylate functional groups per molecule. Other orders of addition may also be practiced to prepare the polyurethane (meth)acrylate, as is known in the art. For example, the hydroxyl-functionalized (meth)acrylate may be first reacted with a polyisocyanate to obtain an isocyanate-functionalized (meth)acrylate, which may then be reacted with an OH group terminated polyester polyol, polyether polyol, polycarbonate polyol, polycaprolactone polyol, polydimethysiloxane polyol, polybutadiene polyol, or a combination thereof. In yet another embodiment, a polyisocyanate may be first reacted with a polyol, including any of the aforementioned types of polyols, to obtain an isocyanate-functionalized polyol, which is thereafter reacted with a hydroxyl-functionalized (meth)acrylate to yield a polyurethane (meth)acrylate Alternatively, all the components may be combined and reacted at the same time.

Suitable acrylic (meth)acrylate oligomers (sometimes also referred to in the art as “acrylic oligomers”) include oligomers which may be described as substances having an oligomeric acrylic backbone which is functionalized with one or (meth)acrylate groups (which may be at a terminus of the oligomer or pendant to the acrylic backbone). The acrylic backbone may be a homopolymer, random copolymer or block copolymer comprised of repeating units of acrylic monomers. The acrylic monomers may be any monomeric (meth)acrylate such as C1-C6 alkyl (meth)acrylates as well as functionalized (meth)acrylates such as (meth)acrylates bearing hydroxyl, carboxylic acid and/or epoxy groups. Acrylic (meth)acrylate oligomers may be prepared using any procedures known in the art, such as by oligomerizing monomers, at least a portion of which are functionalized with hydroxyl, carboxylic acid and/or epoxy groups (e.g., hydroxyalkyl(meth)acrylates, (meth)acrylic acid, glycidyl (meth)acrylate) to obtain a functionalized oligomer intermediate, which is then reacted with one or more (meth)acrylate containing reactants to introduce the desired (meth)acrylate functional groups.

The curable composition of the invention may comprise from 10 to 80 wt. %, in particular from 15 to 75 wt. %, more particularly from 20 to 70 wt. % (meth)acrylate-functionalized oligomer, relative to the entire weight of the free radical curable composition.

For viscosity reasons it might be beneficial that reactive diluents are used. With reactive diluent is meant a compound being able to reduce the viscosity of the formulation while being able to free radically copolymerize. The invention therefore also relates to free radical curable compositions which comprise a reactive diluent. The one or more diluents having one or more free radical curable ethylenically unsaturated groups may be present in the free radical curable composition in an amount of at least 1 wt. %, or at least 5 wt. %, or at least 10 wt. %, and in an amount of at most 85 wt. %, or at most 80 wt. %, or at most 75 wt. %, or at most 70 wt. %, relative to the entire weight of the free radical curable composition.

As reactive diluent various acrylic, methacrylic or vinyl functional monomers can be used.

Suitable examples are for instance diacrylates or dimethacrylates of diols or of polyetherdiols, such as propoxylated neopentyl glycol diacrylate, 1,6-hexanediol diacrylate, dipropylene glycol diacrylate (DPGDA), tripropylene glycol diacrylate (TPGDA), diethylene glycol diacrylate, triethylene glycol diacrylate, triethylenglycol dimethacrylate, neopentylglycol diacrylate, 1,4-butanediol diacrylate (e.g, SR213), alkoxylated aliphatic diacrylate (e.g. SR9209A), alkoxylated hexanediol diacrylate (e.g, SR561, SR562, SR563, SR564 from Sartomer Co., Inc), polyethylene glycol (200) diacrylate (SR259), polyether glycol-200-diacrylate, PEG300-diacrylate, polypropyleneglycol diacrylate, ethoxylated (3) bisphenol-A-diacrylate, BDDA butanediol diacrylate, BDDMA butane diol dimethacrylate or higher functional acrylates such as trimethylolpropane triacrylate (TMPTA), ethoxylated trimethylolpropane triacrylate, TMP3EOTA ethoxylated (3) trimethylolpropane triacrylate, TMP6EOTA ethoxylated (6) trimethylolpropane triacrylate, propoxylated trimethylolpropane triacrylate, pentaerythritol triacrylate, pentaerythritol(4)-propoxylated triacrylate, pentaerythritol tetraacrylate, ethoxylated or propoxylated neopentylglycol, propoxylate (4) glycerol triacrylate, tri-functional monomers, such as Laromer types from BASF or Ebecryl 2047 or Ebecryl 12 from AlInex, di-trimethylolpropane tetraacrylate, dipentaerythritol-pentaacrylate (Di-PEPA), dipentaerythritol hexaacrylate (DPHA). Examples of vinyl compounds are compounds like butanediol divinyl ether or as mono functional compound N-vinyl caprolactam. Although mono functional (meth)acrylates like for example lauryl (meth)acrylate, phenoxyethyl (meth)acrylate can be used as well, it is preferred for low migrateability to employ reactive diluents with at least two free radical curable ethylenically unsaturated groups. Obviously mixtures can be used as well.

In a preferred embodiment of the invention, the oligomeric photoinitiator further comprises one or more free radical curable ethylenically unsaturated groups, preferably one or more (meth)acroyl groups or vinyl groups, more preferably at least two (meth)acroyl groups; and
the free radical curable composition optionally further comprises one or more oligomers having one or more free radical curable ethylenically unsaturated groups, preferably having one or more (meth)acroyl groups or vinyl groups; and
the free radical curable composition further comprises one or more diluents having one or more free radical curable ethylenically unsaturated groups, preferably the one or more diluents having at least two free radical curable ethylenically unsaturated groups; and
the free radical curable composition comprises
   (i) the oligomeric photoinitiator in an amount of from 20 to 70 wt. %,
   (ii) the oligomer in an amount of from 0 to 40 wt. %,
   (iii) the diluent in an amount of from 10 to 60 wt. %,
whereby the amounts are given relative to the entire weight of (i) to (iii).

In accordance with a further aspect, the present invention refers to an free radical curable ink composition or free radical curable coating composition comprising: a) at least one binder, b) at least one ethylenically unsaturated compound selected from monomers and/or oligomers, and one or more oligomeric photoinitiators of the present invention.

If the free radical curable composition is a free radical curable ink composition, it contains at least one pigment. Preferably, the amount of pigment based on the total amount of the free radical curable ink composition, i.e. the composition before curing, is preferably between 0.5 and 50% by weight and more preferably from 3 to 20% by weight. The cured ink composition preferably contains from 0.5 to 50% by weight and more preferably from 3 to 20% by weight of pigment.

In addition to the oligomeric photoinitiator according to the invention, other initiators can be employed as well next to various other additives. Examples for such additives are those selected from the group consisting of rheological additives, adhesion promoters, defoamers, slip additives, wetting agents, levelling agents, gloss additives, waxes, wetting agents, curing agents, chelating agents, additional photoinitiators, amine synergists, inhibitors, desiccants, stabilizers, emulsifiers, abrasion resistance additives, plasticizers, antistatic additives, matting agents and arbitrary combinations of two or more of the aforementioned additives.

The free radical curable ink composition or free radical curable coating composition may be formulated so as to be suitable for any known printing technique, such as offset, lithography, intaglio printing, flexographic printing, gravure printing, screen printing, digital printing, inkjet printing, pad printing, transfer printing, letter printing and the like.

The present invention further relates to a process for free radical curing a free radical curable composition, which comprises the following steps:
(1) Preparing or providing a free radical curable composition as described herein above, and
(2) Free radical curing the free radical curable composition with a light source.

Said light source is preferably an UV light source emitting UV light in at least one of the UVA, UVB and UVC ranges or a LED source emitting light in the range from 350 to 450 nm.

The process preferably comprises the step of applying the free radical curable composition to a substrate prior to free radical curing it.

The present invention further relates to a coating or ink obtained by
(1) Preparing or providing a free radical curable coating or ink composition as described herein above,
(2) Applying the free radical curable coating or ink composition to a substrate, and
(3) Free radical curing the free radical curable coating or ink composition with a light source.

The present invention is now illustrated by reference to the following examples. Unless otherwise specified, all parts, percentages and ratios are on a weight basis.

### Determination of Viscosity

Viscosities were determined on an Anton Paar Physica MCR301 rheometer equipped with 2.5 cm diameter/1° cone/plate geometry at 25° C. at a shear rate of 100 s-1.

### Determination of Molecular Weight by GPC

The number average molecular weight (Mn) and the weight average molecular weight (Mw) were measured via SEC calibrated with a set of polystyrene standards with a molecular weight range of from 500 up to 7×10⁶ g/mol and using as an eluent stabilized tetrahydrofuran [THF with 0.007-0.015% w/w butyl-hydroxytoluene (BHT)] modified with 0.8% acetic acid, at a flow rate of 1 mL/min at 40° C. More specifically, 50 mg of a solid sample of a polyester resin, were dissolved in 5 mL eluent for 16 hours at room temperature without shaking. 10 μL of the solution thus prepared were injected into the system for the measurement. The SEC measurements were carried out on a Waters Acquity® APC™ system which consisted of: i) an Waters Acquity® UPLC RI refractive index detector at 40° C., ii) an Waters Acquity® APC™ Column Manager—S with three different Acquity® APC™ columns (450 Å, 125 Å and 45 Å pore size) with I/d=150/4.6 mm and are filled with particles having a particle size of 2.5 (the 450 Å and the 125 Å column) or 1.7 μm (the 45 Å column) (1 μm=1×10−6 m), (supplied by Waters), iii) an Acquity® APC™ Sample Manager—pFTN injection system and iv) an Acquity® APC™ p-Isocratic Solvent Manager isocratic pump. The Mn and Mw were determined by the use of Empower 3 software from Waters.

### Determination of Photo Reactivity Using Photo DSC Standard Procedure

These analysis were performed using a Mettler Toledo DSC3+ equipped with a Photo DSC fixture bearing an Excelitas Omnicure LX 500 385 nm LED source (1.7 W/cm²) in an nitrogen atmosphere employing around 30 mg formulation.

The following protocol was used: After calibrating the sample for 150 seconds under nitrogen, the sample was irradiated for 150 seconds. This was followed by a dark period of 50 second followed by a 2^{nd} irradiation for 50 seconds. The 2^{nd} irradiation was performed in order to verify that full conversion has already been achieved during the first irradiation. The reactivity is expressed as time to maximum heat-flow (seconds) and in maximum rate of double bond conversion (in mmol s⁻¹ l⁻¹, calculated from the maximum heat-flow (W/g) using 78.5 kJ/mol as reaction enthalpy for acrylates and an assumed density of 1 kg/l).

### Determination of Reactivity Using a UV-Rig

A 100 micron thick film was prepared from the formulations on a glass plate and cured on a Fusion UV Rig equipped with a high power, water cooled 395 nm LED (16 W/cm²) using a total dose of 0.8 J/cm² or a Fusion F600 D bulb (6 W/cm²) using a total dose of 1 J/cm² unless otherwise noted, as determined with an EIT power puck II. After cure the acrylate conversion was determined using IR spectroscopy. The error in the conversion measurement is estimated to be around ±5%.

### Determination of Reactivity Using RT-DMA: Maximum Modulus (G′) and T30%, Modulus Max Values

Values for Maximum Modulus (G′) were determined according to the following procedure described herein. The hardware/equipment used in this procedure was as follows:

### Rheometer+Accessories

ARESG2-rheometer (manufacturer: TA Instruments)
APS temperature control device (Advanced Peltier System)
APS Standard Flat Plate (lower geometry)
ARESG2 UV-curing Accessory (upper plate fixture, UV-light shield back & access door, collimating optic lens)
Ø20 mm acrylic plate with the UV-curing Option upper plate fixture (upper geometry)
Silverline UV radiometer, UV-light sensor (non-calibrated), UV-sensor geometry and disposable plate holder

### UV-Light Source & Other

Omnicure LX500 in combination with 385 nm LED and 8 mm lens attached
Moeller Easy 412-DC-TC Control Relay (trigger box)
UV Power Puck II (Electronic Instrumentation & Technology, calibrated) The hardware described above was then set-up and arranged according to the following. First, UV-curing measurements were performed on the ARESG2 rheometer (TA Instruments). The rheometer was equipped with the APS temperature control device, the APS Standard Flat Plate as lower geometry and the ARESG2 UV-curing Option. The upper geometry used was the upper plate fixture from the ARESG2 UV-curing Option in combination with a 20 mm diameter acrylic plate. As the UV-light source, the Omnicure LX500 spot curing system was used in combination with 385 nm LED (8 mm lens). The 385 nm LED was then inserted into the collimating optic lens of the ARES G2 UV-curing accessory. The collimating lens was fixed to the light shield and aligned to the upper UV geometry mirror and the alignment screws were tightened. The diameter of the original 5 mm lightguide holder part of the collimating lens was increased to 12 mm in order to accommodate the 385 nm UV-LED.

Then, the Omnicure LX500 spot curing system was connected via a Moeller Easy 412-DC-TC Control Relay to the DIGITAL I/O connector at the ARESG2. The Control Relay served as a trigger-box for the UV-light source. The delay time of the trigger was set to 1.5 seconds, meaning that the 385 nm UV-LED was automatically switched on with a delay of 1.5 seconds after the start of the data collection of the modulus measurement on the ARESG2. The light intensity was set to 95%, and the duration of the UV-light was fixed to 128 seconds.

Alignment of the UV-light: Alignment was performed prior to installation of the APS temperature control unit. The UV sensor geometry was attached to a disposable plate holder and installed as the lower geometry. The UV-light sensor, which was connected to Silverline UV-radiometer, was positioned in the outer hole of the UV sensor geometry. The upper geometry was positioned on top of the UV-light sensor by applying approximately 100 grams of axial force. Then, the light intensity was measured at four locations by rotating the lower geometry approximately 90° between each successive measurement. In order to achieve a light distribution at each point which was as equal as possible, the alignment of the collimating lens was then adjusted with the alignment screws on the light shield. The difference in light intensity at the four different positions was maintained to below 10%.

Determination of Light Intensity: Prior to the RT-DMA measurements, the UV-intensity was measured with help of a calibrated UV Power Puck II. To achieve this, the sensor of the UV Power Puck II was positioned directly below the surface of the 20 mm acrylic plate in the upper plate fixture (distance <0.5 mm) with the surface of the acrylic plate completely covering the sensor surface. Next, the Omnicure LX500 UV-source (with an intensity value set to 95%) was manually switched on for 10 seconds. During this 10 second interval, the UVA2 intensity (i.e. radiation between wavelengths of 380-410 nm) was measured with the UV Power Puck II instrument. The measured UVA2 intensity was determined to be between 60-70 mW/cm2, with an actual value of 67 mW/cm2 recorded. Determination of the actual delay time: When starting a measurement, there was a delay between the start of data sampling and the start of UV-illumination. In the settings of the Moeller Easy 412-DC-TC Control Relay, the delay was set to 1.5 seconds, which signifies that the UV-illumination began 1.5 seconds after the initiation of data sampling.

[With help of a Light Dependent Resistance (LDR) and an oscilloscope (PicoScope 3424) an actual delay time of 1.519 s was measured. The delay time of 1.519 seconds was the measured average value of 10 individual measurements with a standard deviation of 0.004 seconds.

[RT-DMA measurement: The RT-DMA UV-curing measurements were then performed using an ARESG2 rheometer paired with the Advanced Peltier System as a temperature control device, the APS Flat Plate, and the ARESG2 UV-curing Accessory set up. A 385 nm LED with an 8-mm lens connected to the Omnicure LX500 was used as the UV light source.

[Sample loading: Prior to loading each respective sample, the temperature of the bottom plate was set to 50° C. When the temperature reached 50° C., the surface of the upper plate (which was an acrylic plate with a thickness of 20 mm) was brought into contact (i.e. a gap of 0 mm with the lower plate by applying an axial force of between 200-400 grams, thereby allowing the upper parallel plate to equilibrate to the set temperature of 50° C. The system was allowed to further equilibrate its temperature for at least 5 minutes after initial contact. Next, a zero-fixture procedure was performed according to well-known methods to determine the gap=0 position. After determining the gap=0 position, the upper plate was moved to a position of 10 mm away. Then a portion of each respective sample was transferred to the center of the lower plate with the tip of a small spatula, after which the upper geometry was lowered to a gap=0.120 mm position. The quantity of the sample had to be sufficient to ensure than an excess would be pushed outside of the gap covering the entire circumference of the upper parallel plate after the upper geometry was brought down to the reduced gap. Next, the excess of sample that had been displaced outside of the gap was removed, and the upper geometry was brought down further to the measuring position (having a gap=0.100 mm). With the measuring position loaded, the temperature of the sample was allowed to equilibrate to 50° C. Finally, when the sample temperature was measured as stable between 49.9° and 50.10° C., the measurement process would commence by activating the trigger box (Moeller Easy 412-DC-TC) and using the interface and interconnection provided by the TRIOS software package. [Measurement: The actual UV cure RT-DMA measurement was a so-called “fast sampling” measurement taken at 50° C. That is, it was an oscillation fast sampling taken at 50° C. for a duration of 128 seconds, with a 1% strain, a rotational velocity of 52.36 rad/s, and a measurement frequency of 50 points per second (i.e. 0.020 seconds between each successive measurement point).

Then, the measurement was started via the start button in the TRIOS software. Once the data sampling started, the rheometer sent a signal to the control relay, which in turn activated the Omnicure LX500 UV-light source to illuminate the respective sample with the aforementioned delay of 1.519 s after commencement of data sampling. The sample was illuminated with the 385 nm UV-light (Intensity 70 mW/cm2) during 128 seconds of fast sampling data collection as described above. After the measurement was finished, the TRIOS data file was exported to Microsoft Excel. Then the sample was removed and the plates subsequently cleaned thoroughly with ethanol prior to loading of the next sample. Data analysis: As mentioned, the TRIOS data was exported to Microsoft Excel. Excel was used to plot graphs and calculate various parameters for characterization of the cure speed performance of the tested formulations as described below. The graphs included those corresponding to storage modulus (G′) as a function of UV-time (UV-time was calculated by subtracting the delay time (1.519 s) from the actual time for each individual data point), and relative storage modulus (rel G′) as function of UV-time (rel G′ was calculated by the quotient of the measured G′ value at certain UV-time and the maximum obtained G′ value during the cure measurement). The maximum value observed of the graph of the G′ graph was determined by taking the average of the G′ value between 110 and 120 seconds, and is reported in Table 6 below under the column headed by “Max. G′”.

The characteristic parameters, meanwhile, included: (1) the time to reach 30% of the total storage modulus (G′) increase, and (2) average G′ 110-120 s (Average storage modulus value out of 6 datapoints towards the end of the cure measurement). The results for (1) of each formulation is reported in Table 6 below under the column headed by T30%, modulus max.

### EXAMPLES

These examples illustrate embodiments of the instant invention. Table 1 describes the various components used to prepare the photoinitiators and the free radical curable compositions used in the present examples. Table 2 describes the relative amounts of the reagents described in Table 1 which was used to synthesize the photoinitiators used in the present examples. Tables 3 to 6 reports the photoreactivity of the photoinitiators.

TABLE 1
Components to prepare the photoinitiators
and the free radical curable compositions

| Compo- | | Supplier/ |
|---|---|---|
| nent | Chemical Description | Manufacturer |
| AdA | Adipic acid; CAS no 124-04-9 | BASF |
| KGA | Ketoglutaric acid; CAS no 328-50-7 | Sigma Aldrich |
| POG | Propoxylated glycerol (PO)5; CAS no | |
| | 25791-96-2 | |
| HD | Hexane diol; CAS no 629-11-8 | BASF |
| PA | Pyruvic acid; CAS no 127-17-3 | Sigma Aldrich |
| AA | Acrylic acid; CAS no 79-10-7 | Evonik |
| KAoi | Isocyanato ethyl acrylate; CAS no | Showa-Denko |
| | 13641-96-8 | |
| | Methoxyphenol; CAS no 150-76-5 | Sigma Aldrich |
| BHT | Butylated Hydroxy toluene; Cas | Sigma Aldrich |
| | no 128-37-0 | |
| | Toluene; CAS no 108-88-3 | Exon mobile |
| MSA | Methanesulphonic acid; CAS no | BASF |
| | 75-75-2 | |
| Epikote ™ | Epoxy resin Mₙ 350; CAS no | Hexion Speciality |
| 828 | 68038-82-4 | Chemicals Inc. |
| DBTDL | Dibutyl tin dilaurate; CAS no | Valtris Speciality |
| | 77-58-7 | Chemicals Ltd. |
| | Bismuth neodecanoate; CAS no | Sigma Aldrich |
| | 343-26-6 | |
| Neorad | Urethane acrylate resin diluted with | Covestro |
| U25-20D | HDDA | |
| DMAB | Ethyl 4-N,N-dimethyl amino benzoate; | Sigma Aldrich |
| | CAS 10287-53-3, amine synergist | |
| BP | Benzophenone; CAS no 119-61-9 | Sigma Aldrich |
| Acclaim | Bifunctional polypropylene glycol; | Covestro |
| 4200 | molecular weight approximately 4000 | |
| (PPG4000) | | |
| TDI | Toluenediisocyanate; CAS no 584-84-9 | Covestro, BASF |
| HEA | 2-hydroxyethyl acrylate; CAS no | Nippon Shokubai |
| | 818-61-1 | |
| HHPZA | Hexahydrophatlic anhydride: CAS no | Sigma Aldrich |
| | 85-42-7 | |
| TMPO | Trimethylolpropane oxetane; CAS no | Sigma Aldrich |
| | 3047-32-3 | |
| HMwPOG | Propoxylated (PO)17 glycerol; | Dow Chemical |
| | Voranol ™ CP1055; CAS no | Company |
| | 25791-96-2 | |

### Synthesis of Oligomeric Photoinitiator OPI 1-8 (Table 2)—Typical Procedure

### OPI 1:

Step 1) A 500 ml reactor equipped with a stirrer, nitrogen inlet, and Dean-Stark set-up was charged with 16.7 g adipic acid (AdA), 102.9 g propoxylated glycerol (POG), 240 g toluene and 0.74 g methane sulphonic acid MSA. The reaction was heated to reflux under a gentle stream of nitrogen and kept at this temperature until reaction water was no longer formed (4 hr).
Next (Step 2) 40.2 g Pyruvic acid was added and the reaction mixture was heated to reflux under a gentle stream of nitrogen and kept at this temperature until reaction water was no longer formed (4 hr). Next all the toluene was removed by distillation under reduced pressure at a temperature of 95° C. Next 2.7 g Epikote™ 828 was added and the reaction was stirred for another 30 min, cooled down after which OPI 1 was obtained having a viscosity of 4 Pa·s at 100 s⁻¹.

The synthesis of oligomeric photoinitiators OPI 2-8 was performed similar to OPI1, except for the amounts of the components as reported in Table 2.

For example the structural formula for OPI5 with idealized structure (KGA)₁(POG)₂(PA)₄ is

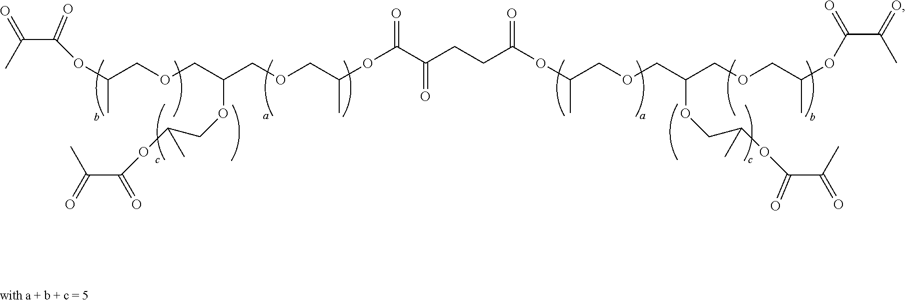

### Synthesis of Acrylate Functional Oligomeric Photoinitiator OPI 9 and OPI 10

OPI9: This synthesis was performed similar to OPI 7 except that in step 2 24.9 g pyruvic acid, 4.1 g acrylic acid and 0.07 g methoxy phenol were added instead of 29.9 g pyruvic acid.
OPI10: similar to OPI 9 using the amounts as reported in table 2 with 0.07 g methoxy phenol.

### Synthesis Urethane Acrylate Functional Oligomer Photo Initiator OPI11

A 100 ml reactor equipped with a mechanical stirrer and a dry air inlet was charged with 20 g of OPI 7 and 0.05 g DBTDL. Next, under vigorous stirring and a gentle dry air flow was added 1.1 g KAoi. The reaction mixture was heated under stirring to 60° C. and kept at this temperature until the isocyanate was completely reacted (around 30 min, no NCO visible in IR) resulting in urethane acrylate functional oligomer photo initiator OPI 11. The idealized structure is (KGA)₍₃₋ₓ₎(POG)₄(PA)_{(6-y)}KAoi_{(x+y=1)} due to the fact that both α-keto glutarate and pyruvate can enolize.

### Synthesis of Oligomeric Photo Initiator OPI12

Step 1) A 11 reactor equipped with a stirrer, nitrogen inlet, and cooler was charged with 249.7 g hexahydrophthalic acid (HHPZA), 250.1 g trimethylol propane oxetane (TMPO) and 0.250 g sodium acetate (500 ppm) The reaction was heated to 170° C. under a gentle stream of nitrogen and kept at this temperature for 17 hr. When the acid value was below 20, the resin was discharged.
Step 2) A 500 ml reactor equipped with a stirrer, nitrogen inlet, and Dean Stark set-up was charged with 92.1 g of the hydroxy functional resin from step 1, 52.6 g pyruvic acid, 0.69 g MSA and 221.8 g toluene. The reaction was heated to reflux under a gentle stream of nitrogen and kept at this temperature until reaction water was no longer formed (6 hr).

Next all the toluene was removed by distillation under reduced pressure at a temperature of 95° C. Next 2.5 g Epikote™ 828 was added and the reaction was stirred for another 30 min, cooled down after which OPI12 was obtained as a solid.

### Synthesis of Oligomeric Photo Initiator OPI13

A 500 ml reactor equipped with a stirrer, nitrogen inlet, and Dean-Stark set-up was charged with 123.7 g high Mw propoxylated glycerol (Voranol CP1055, PO17), 32.7 g pyruvic acid, 240 g toluene and 0.77 g methane sulphonic acid MSA. The reaction was heated to reflux under a gentle stream of nitrogen and kept at this temperature until reaction water was no longer formed (4 hr). Next all the toluene was removed by distillation under reduced pressure at a temperature of 95° C. Next 2.7 g Epikote™ 828 was added and the reaction was stirred for another 30 min, cooled down after which OPI 13 was obtained having a viscosity of 0.33 Pa·s at 100 s⁻¹

### Synthesis Comparative Linear Oligomeric Example (Liska) Comp L1

The synthesis has been performed analogous to (R. Taschner, P. Gauss, P. Knaack, R. Liska, “Biocompatible Photoinitiators Based on Poly-α-ketoesters”, Journal of Polymer Science vol 58, p 242-253, (2020) except on similar scale to OPI 1-9) using 100.6 g α-ketoglutaric acid, 89.5 g hexanediol (1.1 molar equivalents), 270 g toluene and 0.7 g MSA. The resulting solid comparative initiator L1 had a Mₙ of 2.6 kg/mol and an M_{w} of 9.2 kg/mol. Tm 46-54° C. (1.1 eq)

### Synthesis Comparative Linear Oligomeric Example (Liska) Comp L2

The procedure for L1 was repeated using 98.9 g α-ketoglutaric acid, 80.70 g hexanediol (1.01 molar equivalents, analogous to US 2020/0231531), 220 g toluene and 0.26 g MSA. After precipitation and evaporation of the remaining solvent L2 was obtained as white powder. The resulting comparative initiator L2 had a Mₙ of 5.1 kg/mol and an M_{w} of 17.2 kg/mol. Tm 40-51° C. (1.01 eq)

### Synthesis Comparative Linear Oligomeric Example (Liska) Comp L3

The procedure for L1 was repeated using 100.6 g α-ketoglutaric acid, 78.6 g hexanediol (0.96 molar equivalents), 270 g toluene and 0.7 g MSA. Employing this equivalency an acid functional linear polyester was prepared. The resulting solid comparative initiator L3 had a Mₙ of 3.4 kg/mol and a M_{w} of 12.1 kg/mol. Tm 52-59° C. (0.96 eq)

TABLE 2
Amount used in the synthesis and data of the resulting OPI's

| | | | Step 2 | | | | | | |
|---|---|---|---|---|---|---|---|---|---|
| | Step 1 | | Mono | Mono | | Viscosity | Mₙ | | |
| | Diacid | Triol | acid | acid | | Pa · s25° | Theo | Mₙ | M_{w} |
| OPI | (g) | (g) | 1(g) | 2 (g) | Idealized structure | C./100 s⁻¹ | Da | kDa | kDa |
| 1 | AdA | POG | PA | | (AdA)₁(POG)₂(PA)₄ | 4 | 1291 | 1.4 | 2.8 |
| | 16.7 | 102.9 | 40.2 | | | | | | |
| 2 | AdA | POG | PA | | (AdA)₂(POG)₃(PA)₅ | 5.7 | 1922 | 1.6 | 3.7 |
| | 22.2 | 102.9 | 33.5 | | | | | | |
| 3 | AdA | POG | PA | | (AdA)₃(POG)₄(PA)₆ | 8.5 | 2522 | 1.8 | 5.3 |
| | 24.8 | 101.9 | 29.9 | | | | | | |
| 4 | AdA | POG | PA | | (AdA)₁₅(POG)₁₆(PA)₁₈ | 81 | 10119 | 2.6 | 35.8 |
| | 31.2 | 102.7 | 22.6 | | | | | | |
| 5 | KGA | POG | PA | | (KGA)₁(POG)₂(PA)₄ | 5.7 | 1291 | 1.2 | 2.3 |
| | 16.3 | 100.8 | 39.4 | | | | | | |
| 6 | KGA | POG | PA | | (KGA)₂(POG)₃(PA)₅ | 12 | 1922 | 1.4 | 3.4 |
| | 22 | 101.5 | 33.1 | | | | | | |
| 7 | KGA | POG | PA | | (KGA)₃(POG)₄(PA)₆ | 25 | 2552 | 1.7 | 4.7 |
| | 24.8 | 101.8 | 29.9 | | | | | | |
| 8 | KGA | POG | PA | | (KGA)₁₅(POG)₁₆(PA)₁₈ | 147 | 10118 | 2.2 | 15.8 |
| | 31.3 | 102.7 | 22.6 | | | | | | |
| 9 | KGA | POG | PA | AA | (KGA)₃(POG)₄(PA)₅(AA)₁ | 25 | 2536 | 1.7 | 4.7 |
| | 24.8 | 101.8 | 24.9 | 4.1 | | | | | |
| 10 | KGA | POG | PA | AA | (KGA)₃(POG)₄(PA)₄(AA)₂ | 24 | 2448 | 1.6 | 5.7 |
| | 25.1 | 103 | 20.1 | 8.2 | | | | | |
| 11 | | | | | (KGA)₍₃₋ₓ₎(POG)₄(PA)_{(6-y)} | 57 | 2693 | 1.8 | 5.7 |
| | | | | | KAOi_{(x+y=1)} | | | | |
| 12 | HHPZA | TMPO | PA | | (HHPZA)₃(TMPO)₄(PA)₆ | x | 1828 | 1.2 | 2.4 |
| | 249.7 | 250.1 | 52.6 | | | | | | |
| 13 | | HMwPOG | PA | | (HMwPOG)₁(PA)₃ | 0.33 | 1288 | 1.2 | 1.6 |
| | | 123.7 | 32.7 | | | | | | |

x: solid, melting point of 16-29° C.

### Synthesis of Urethane Oligomer 1

First, the reactor was purged with dry lean air. Then 2.19 parts of BHT was added before 52.76 parts of TDI followed by 0.02 parts of acrylic acid were charged into a reactor (equipped with a stirrer, air inlet, dropping funnel, and condenser). After charging, the reactor was heated to 45° C. Then half of the specified amount catalyst (eg 0.06 g bismuth neodecanoate) followed by 35.43 parts HEA were charged into the reactor whilst stirring. After waiting one (1) hour for the reaction to commence, the temperature was then raised to 60° C. At 60° C., 700 parts PPG4000 and the second part of the catalyst (eg 0.07 g) were added after which the reaction temperature was raised to 85° C. then further maintained for two (2) additional hours.

After this two (2) additional hours of reaction time, the quantity of isocyanate (NCO) content was measured by a potentiometric titrator to ensure it was lower than 0.1% relative to the entire weight of the composition. If the isocyanate content was not lower than this value, the mixture was placed back in the reaction chamber in 15-minute additional increments (again at 85° C.) and checked again, with this step repeated until the isocyanate content fell to within the desired range. Finally, the resulting synthesized oligomer was cooled slowly and discharged for use. The resulting oligomer 1 with the idealized structure HEA-TDI-PPG4000-TDI-HEA.

### Preparation of Formulations

Each of the formulations described in Tables 3-6 below were prepared by conventional methods by using a 50 ml mixing cup suitable for use with a Speedmixer™. The components, adding up to 10 g in total, were added in the mixing cup. The cup was then closed and vigorously mixed in a Speedmixer™ DAC150FVZ for 5 minutes, stopped, and mixed again for 5 additional minutes via the same method.

### Examples 1-2 and Comparative Experiment A

Formulations were prepared in the speed mixer using 99.5 parts Neorad U25-20D and 0.5 parts of various initiators. Curing was performed on the UV rig. The results are in table 3 below.

TABLE 3
UV rig curing

| | | mmol | Conversion | Conversion |
|---|---|---|---|---|
| | | chromophore/ | with 395 nm | with D bulb |
| Example | Initiator | kg | LED (%) | (%) |
| 1 | OPI 1 | 15.5 | 62 | 87 |
| 2 | OPI 5 | 19.4 | 88 | 92 |
| Comparative A | L1 | 20.3 | 5 | 65 |

These examples clearly demonstrate that the structures according to the current invention yield a more efficient cure process compared to the linear polymer L1. The high efficiency is especially surprising when comparing the amount of active chromophores present in the formulations. Even with less active chromophores, the curing proceeds much more efficient using the OPI's according to the invention.

This efficient curing is further illustrated by employing 0.5 parts benzophenone (27.4 mmol chromophores/kg) and 0.2 parts ethyl 4-N,N-dimethyl amino benzoate (amine synergist) being a well-known type II initiating system. After curing with the D-bulb a conversion of 82% was observed.

### Examples 3-15 and Comparative Experiments B-D

Formulations were prepared in the speed mixer using 70 parts urethane oligomer 1, 30 parts phenoxy ethyl acrylate and 0.5 parts of various initiators. Curing was performed using photo-DSC employing a 385 nm LED. The results are shown in table 4.

TABLE 4
Photo-DSC curing

| | | | | Time to |
|---|---|---|---|---|
| | | | Maximum | maximum |
| Exam- | | | rate: | heat |
| ple | OPI | Idealized structure | (mmol s⁻¹ l⁻¹) | flow (s) |
| 3 | 1 | (AdA)₁(POG)₂(PA)₄ | 199 | 10.7 |
| 4 | 2 | (AdA)₂(POG)₃(PA)₅ | 194 | 10.9 |
| 5 | 3 | (AdA)₃(POG)₄(PA)₆ | 194 | 11.3 |
| 6 | 4 | (AdA)₁₅(POG)₁₆(PA)₁₈ | 137 | 14.7 |
| 7 | 5 | (KGA)₁(POG)₂(PA)₄ | 187 | 10.9 |
| 8 | 6 | (KGA)₂(POG)₃(PA)₅ | 180 | 10.9 |
| 9 | 7 | (KGA)₃(POG)₄(PA)₆ | 182 | 10.9 |
| 10 | 8 | (KGA)₁₅(POG)₁₆(PA)₁₈ | 177 | 11.3 |
| 11 | 9 | (KGA)₃(POG)₄(PA)₅(AA)₁ | 180 | 11.3 |
| 12 | 10 | (KGA)₃(POG)₄(PA)₄(AA)₂ | 180 | 13.0 |
| 13 | 11 | (KGA)₍₃₋ₓ₎POG)₄(PA)_{(6-y)} | 180 | 10.8 |
| | | KAoi_{(x+y=1)} | | |
| 14 | 12 | (HHPZA)₃(TMPO)₄(PA)₆ | 195 | 10.8 |
| 15 | 13 | HMwPOG(PA)₃ | 181 | 10.8 |
| B | | L1 (1.1 eq) ((HD)(KGA))ₙ | 108 | 18.8 |
| C | | L2 (1.01 eq) | 123 | 14.4 |
| | | ((HD)(KGA))ₙ | | |
| D | | L3 (0.96 eq) | 105 | 17.5 |
| | | ((HD)(KGA))ₙ | | |

Again this demonstrates the surprisingly high efficiency of the oligomeric photoinitiators according to the invention compared to L1-L3

### Examples 16-26 and Comparative Experiments E-G

Formulations were prepared in the speed mixer using 70 parts urethane oligomer 1, 30 parts phenoxy ethyl acrylate and 5 parts of various initiators. Curing was performed on the UV rig using 395 nm LED employing a total dose of only 0.4 J/cm2. This low dose was taken to ensure that full conversion was not reached.

For the comparative examples the formulations had to be heated to 50° C. before mixing in the speed mixer. Otherwise no clear formulation was obtained.

TABLE 5
Reactivity on the UV rig using 395 nm LED

| | | | Acrylate |
|---|---|---|---|
| Example | OPI | | Conversion (%) |
| 16 | 1 | (AdA)₁(POG)₂(PA)₄ | 49 |
| 17 | 2 | (AdA)₂(POG)₃(PA)₅ | 56 |
| 18 | 3 | (AdA)₃(POG)₄(PA)₆ | 54 |
| 19 | 4 | (AdA)₁₅(POG)₁₆(PA)₁₈ | 41 |
| 20 | 5 | (KGA)₁(POG)₂(PA)₄ | 49 |
| 21 | 6 | (KGA)₂(POG)₃(PA)₅ | 53 |
| 22 | 7 | (KGA)₃(POG)₄(PA)₆ | 49 |
| 23 | 8 | (KGA)₁₅(POG)₁₆(PA)₁₈ | 44 |
| 24 | 9 | (KGA)₃(POG)₄(PA)₅(AA)₁ | 51 |
| 25 | 10 | (KGA)₃(POG)₄(PA)₄(AA)₂ | 47 |
| 26 | 11 | (KGA)₍₃₋ₓ₎POG)₄(PA)_{(6-y)} | 48 |
| | | KAoi_{(x+y=1)} | |
| E | | L1 (1.1 eq) ((HD)(KGA))ₙ | 27 |
| F | | L2 (1.01 eq) ((HD)(KGA))ₙ | 38 |
| G | | L3 (0.96 eq) ((HD)(KGA))ₙ | 24 |

### Examples 27-38 and Comparative H-K Reactivity RT-DMA

Formulations were prepared in the speed mixer using 70 parts urethane oligomer 1, 30 parts phenoxy ethyl acrylate and 5 parts of various initiators. Curing was performed on the in the RT-DMA using 385 nm LED with an intensity of 70 mW/cm2 For the comparative examples the formulations had to be heated to 50° C. before mixing in the speed mixer. Otherwise no clear formulation was obtained.

With amine, formulations were prepared with 70 parts urethane oligomer 1, 30 parts phenoxy ethyl acrylate and 5 parts of various initiators and 5 parts ethyl 4-N,N-dimethyl amino benzoate.

TABLE 6

| | | | Max | T30%, |
|---|---|---|---|---|
| | | | G′ | modulus |
| Example | OPI | | (MPa) | max (s) |
| 27 | 1 | (AdA)₁(POG)₂(PA)₄ | 0.57 | 30 |
| 28 | 2 | (AdA)₂(POG)₃(PA)₅ | 0.59 | 29 |
| 29 | 3 | (AdA)₃(POG)₄(PA)₆ | 0.58 | 32 |
| 30 | 4 | (AdA)₁₅(POG)₁₆(PA)₁₈ | 0.50 | 54 |
| 31 | 5 | (KGA)₁(POG)₂(PA)₄ | 0.60 | 24 |
| 32 | 6 | (KGA)₂(POG)₃(PA)₅ | 0.58 | 25 |
| 33 | 7 | (KGA)₃(POG)₄(PA)₆ | 0.60 | 27 |
| 34 | 8 | (KGA)₁₅(POG)₁₆(PA)₁₈ | 0.56 | 38 |
| 35 | 9 | (KGA)₃(POG)₄(PA)₅(AA)₁ | 0.56 | 29 |
| 36 | 10 | (KGA)₃(POG)₄(PA)₄(AA)₂ | 0.56 | 32 |
| 37 | 11 | (KGA)₍₃₋ₓ₎POG)₄(PA)_{(6-y)} | 0.57 | 36 |
| | | KAoi_{(x+y=1)} | | |
| H | | L1 (1.1 eq) | 0.19* | 78** |
| I | | L2 (1.01 eq) | 0.42* | 50** |
| J | | L3 0.96 eq acid funct | 0.17* | 70** |
| 38 | | (KGA)₁(POG)₂(PA)₄ with amine | 0.50 | 18 |
| K | | L2 with amine | 0.43* | 32** |

*Plateau value not reached, incomplete cure modulus to low.
**value based on modulus @ 120 sec.

1. An oligomeric photoinitiator according to formula (I) 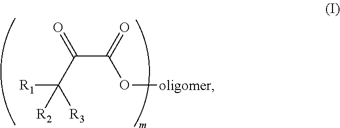
wherein
m is equal to or greater than 3,
R₁, R₂ and R₃ are independently selected from H or from a linear or branched C₁-C₆ alkyl, and
wherein the oligomeric photoinitiator has a weight-average molecular weight M_{w} as determined by GPC (THF) higher than 1000 g/mol, whereby the weight-average molecular weight M_{w} is determined as described in the description.

2. The oligomeric photoinitiator according to claim 1, wherein the weight-average molecular weight M_{w} of the oligomeric photoinitiator is greater than 1250 g/mol.

3. The oligomeric photoinitiator according to claim 1, wherein R₁, R₂ and R₃ are independently selected from H or CH₃.

4. The oligomeric photoinitiator according to claim 1, wherein m is equal to or greater than 5.

5. The oligomeric photoinitiator according to claim 1, wherein the oligomeric photoinitiator comprises from 1 to 10 groups according to formula (II) 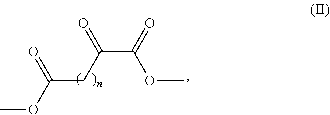
wherein n is from 0 to 6.

6. A process for preparing the oligomeric photoinitiator according to claim 1 comprising:
(1) providing a core oligomer having at least m terminal OH functional groups, wherein m has a value of ≥3, and
(2) (trans)esterifying the m terminal OH functional groups of the core oligomer with a compound according to formula (Ib): 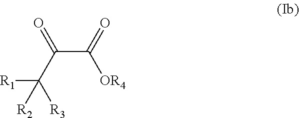
wherein R₄ is H or a C₁-C₄ alkyl; and R₁, R₂ and R₃ are independently selected from H or from a linear or branched C₁-C₆ alkyl.

7. The oligomeric photoinitiator according to claim 1, wherein the oligomeric photoinitiator comprises free radical polymerizable groups.

8. The oligomeric photoinitiator according to claim 7, wherein the free radical polymerizable groups are selected from the group consisting of methacrylates, acrylates, vinyl amides, vinyl ethers, vinyl esters, fumarates, itaconates, maleates and any combination thereof.

9. The oligomeric photoinitiator according to claim 1, wherein the oligomer is highly branched, more preferably hyperbranched.

10. A free radical curable composition comprising at least one oligomeric photoinitiator according to claim 1.

11. The free radical curable composition according to claim 10, wherein the oligomeric photoinitiator is present in an amount of from 0.05 to 30 wt. %, relative to the entire weight of the free radical curable composition.

12. The free radical curable composition according to claim 10, wherein the free radical curable composition further comprises one or more oligomers having one or more free radical curable ethylenically unsaturated groups.

13. The free radical curable composition according to claim 12, wherein the one or more oligomers having one or more free radical curable ethylenically unsaturated groups are independently selected from urethane (meth)acrylate oligomers, epoxy (meth)acrylate oligomers, polyether (meth)acrylate oligomers, and polyester (meth)acrylate oligomers.

14. The free radical curable composition according to claim 12, wherein the one or more oligomers having one or more free radical curable ethylenically unsaturated groups have a number average molecular weight (Mₙ) equal to or higher than 600 g/mol, in which the number-average molecular weight Mₙ is determined as described in the description.

15. The free radical curable composition according to claim 12, wherein the one or more oligomers having one or more free radical curable ethylenically unsaturated groups are present in the free radical curable composition in an amount of at least 10 wt. %, relative to the weight of the entire free radical curable composition.

16. The free radical curable composition according to claim 10, wherein the free radical curable composition further comprises one or more diluents having one or more free radical curable ethylenically unsaturated groups.

17. The free radical curable composition according to claim 16, wherein the one or more diluents having one or more free radical curable ethylenically unsaturated groups are present in the free radical curable composition in an amount of at least 1 wt. %, relative to the entire weight of the free radical curable composition.

18. The free radical curable composition according to claim 10, wherein
the oligomeric photoinitiator further comprises one or more free radical curable ethylenically unsaturated groups;
the free radical curable composition optionally further comprises one or more oligomers having one or more free radical curable ethylenically unsaturated groups; and
the free radical curable composition further comprises one or more diluents having one or more free radical curable ethylenically unsaturated groups; and wherein
the free radical curable composition comprises:
(i) the oligomeric photoinitiator in an amount of from 20 to 70 wt. %,
(ii) the one or more oligomers in an amount of from 0 to 40 wt. %,
(iii) the one or more diluents in an amount of from 10 to 60 wt. %,
in which the amounts are given relative to the entire weight of (i) to (iii).

19. The free radical curable composition according to claim 10, wherein the free radical curable composition is a coating or ink composition.

20. A process for preparing a coating or ink comprising:
(1) preparing or providing a free radical curable coating or ink composition according to claim 19,
(2) applying the free radical curable coating or ink composition to a substrate, and
(3) free radical curing the free radical curable coating or ink composition with a light source.

21. The oligomeric photoinitiator according to claim 1, wherein R₁, R₂ and R₃ are H.

22. The oligomeric photoinitiator according to claim 5, wherein n is 2.

23. The oligomeric photoinitiator according to claim 7, wherein the free radical polymerizable groups are methacrylates and/or acrylates.

24. The free radical curable composition according to claim 10, wherein the oligomeric photoinitiator is present in an amount of from 0.1 to 15 wt. %, relative to the entire weight of the free radical curable composition.
